(19) United States
(12) Patent Application Publication
(10) Pub. No.: US 2013/0018061 A1
(43) Pub. Date: Jan. 17, 2013

(54) Method for Improving Exercise and Recovery From Exercise

(71) Applicants: Raza Bashir, Oakville (CA); Craig Wissent, Oakville (CA)

(72) Inventors: Raza Bashir, Oakville (CA); Craig Wissent, Oakville (CA)

(73) Assignee: Northern Innovations and Formulations Corp, Oakville (CA)

(21) Appl. No.: 13/180,872

(22) Filed: Jul. 12, 2011

(51) Int. Cl. A61K 31/522; A61P 43/00

(52) U.S. Cl. 514/263.31

(57) ABSTRACT

A method for improving exercise performance and recovery from exercise is provided. The method involves administration of a specific ratio of caffeine and L-theanine. The method is useful for any individual participating in exercise and may be particularly beneficial for individuals accustomed to regular, high caffeine intake.

### FIELD OF THE INVENTION

The present invention relates generally to methods for improving exercise performance and recovery from exercise. More specifically, the invention provides methods comprising administration of caffeine and L-theanine in a specific ratio effective for improving exercise performance and recovery from exercise.

### BACKGROUND OF THE INVENTION

Individuals, whether athletes or recreationally active, often seek methods to improve exercise performance. ‘Pre-workout’ supplements are a class of nutritional supplements taken with the goal of improving or enhancing performance. Traditional pre-exercise strategies focused on carbohydrate administration while more contemporary research has suggested the benefits of amino acids, protein, creatine, and caffeine.

Energy drinks may, in some cases, be considered as pre-workout supplements when used before exercise, however their use is often more prevalent as a cognitive enhancer or an anti-fatigue supplement for mental or attentional improvement, particularly to combat lack of sleep.

In any case, caffeine is a popular ingredient alone or as part of pre-workout supplements to provide energy and focus during exercise. Such use of caffeine is primarily due to its action as a central nervous system stimulant but may also spare glycogen by promoting the utilization of fat for energy.

Caffeine is possibly the most extensively consumed psychoactive substance in the Western hemisphere. In addition to being a central nervous system stimulant, it has a variety of peripheral effects relating to muscle contraction, gastric secretion, and lipolysis. Chemically, caffeine (1, 3, 7-trimethylxanthine) is a member of a class of compounds known as methylxanthines. Caffeine is easily absorbed in the gastrointestinal tract, to the blood stream and metabolized in the liver. Plasma levels of caffeine increase quickly and peak at about one hour post-ingestion with a half-life of 4 to 6 hours. In adults, 1 to 5% of ingested caffeine is excreted unchanged in the urine, while the other 95 to 99% is metabolized and excreted as conjugates.

Caffeine's main mechanism is mediated through selective antagonism of the adenosine A1 and A2A receptors. Adenosine is a cellular constituent that modulates the release and activity of a variety of neurotransmitters, including norepinephrine, acetylcholine, dopamine, and GABA. It primarily inhibits the release of excitatory neurotransmitters, so increased concentrations of adenosine reduce arousal and suppress spontaneous behavioral activity. Adenosine concentrations slowly increase during wakefulness, so adenosine antagonism is what causes caffeine's wake-promoting effect.

Despite its widespread use, there are some drawbacks associated with the use, particularly chronic use, of caffeine such as tolerance and sensitivity and side effects that may be associated with caffeine consumption including ‘jolt and crash’, increased cortisol, insomnia and jitteriness. It is known that habitual caffeine use can lead to tolerance whereby the stimulant effects of an acute dose of caffeine are diminished. Tolerance—real or perceived—to some of the effects of caffeine with regular use develops quickly, typically from hours to days and is associated with an increase in adenosine receptor activity. This tolerance is related to withdrawal symptoms. However, despite tolerance, benefits from regular caffeine consumption persist when used in the context of pre-workout supplementation. Despite any unwanted effects, caffeine continues to be regular component of daily dietary consumption and a popular pre-workout aid for many individuals.

L-Theanine (γ-N-ethylglutamine) is a unique amino acid found in green tea and mushrooms. Absorbed in the small intestine, it crosses the blood-brain barrier and into the brain in a dose-dependent manner, with peak plasma levels appearing within about 30 minutes. L-theanine is a well-studied compound and has been shown to stimulate the production of alpha brain waves (a state often achieved by meditation—one of deep relaxation and mental alertness) likely in association with increasing serotonin and dopamine production.

Research shows L-theanine and caffeine in combination are beneficial for improving performance on cognitively demanding tasks to a greater extent than caffeine alone. One particular study examined the effects of 250 mg caffeine, 200 mg L-theanine, or both on anxiety, alertness, and blood pressure. It was found that caffeine increased alertness, anxiety, and blood pressure, while L-theanine only acted to counter the increase in blood pressure. Rogers et al., Psychopharmacology, 195:569-577 (2008) [See also, Haskell et al., Biol. Psych, 77:113-122 (2007); Perrini, L-Theanine: How a Unique Anxiety Reducer and Mood Enhancer Increases Alpha Waves (http://web-us.com/I-theanine_anxiety_reducer.htm#top); “L-theanine plus caffeine enhances cognitive performance” (http://www.vrp.com/brain-health/l-theanine-plus-caffeine-enhances-cognitive-performance)]

There has been little research done on L-theanine in the context of exercise. One study examined the effects of L-theanine on improving mental regeneration after physical exercise. Fourteen healthy male subjects were given 50 mg or 200 mg of L-theanine after cycling exercise and it was found that L-theanine accelerated post-exercise mental regeneration via a mechanism that was not dose-dependent. A group of studies examined the effects of a supplement containing 700 mg cysteine and 280 mg L-theanine or placebo given daily to male runners for 7 or 10 days prior to and during a 9 or 10 day training camp. The results showed that the cysteine/L-theanine combination attenuated impaired immune function resulting from endurance exercise [Jaeger et al., J. International Society of Sports Nutrition, 5 Supp. 1:P3 (2008)].

US Application 2005/0020627 describes the use of L-theanine for improving mind-concentration during physical exercise. No suggestion is made to combine with caffeine, which is only referred to as a drug with negative effects.

U.S. Pat. No. 5,736,575 describes the use of L-theanine as an “excitement accelerating agent”. The only disclosure with regard to caffeine is that L-theanine suppresses its stimulant activity and may be used for that purpose in caffeine-containing foods or drinks to maintain taste and flavor.

U.S. Pat. No. 6,462,051 describes a composition comprising caffeine, L-theanine, and arginine in a ratio where the caffeine is less than 1:1:1 for reducing mental fatigue or enhancing concentration. There is no disclosure regarding the activity or interactions of the ingredients.

The inventors identified a composition and method to reduce some of the unwanted effects associated with the pre-workout consumption of caffeine and while maintaining or improving upon its usefulness as an ergogenic aid. To the knowledge of the inventors the effects of the specific combination of caffeine and L-theanine herein disclosed in the context of pre-workout supplementation has been heretofore unknown.

### SUMMARY OF THE INVENTION

The invention provides methods and compositions for improving exercise performance comprising caffeine and L-theanine in a ratio of about 3:2. In certain embodiments the composition is administered from about 30 minutes to 45 minutes prior to the commencement of exercise. Accordingly, the method may comprise administering a composition comprising from about 90 to 450 mg of caffeine per serving; and from about 60 to about 300 mg of theanine per serving; preferably from about 150 to 375 mg of caffeine per serving, and from about 100 to 250 mg theanine, maintaining a ratio of about 3:2 caffeine:theanine. In certain embodiments the composition comprises about 300 mg caffeine and about 200 mg L-theanine per serving.

According to another aspect, methods and compositions for improving recovery from exercise are provided. In certain embodiments the composition is administered from about 30 minutes to 45 minutes prior to the commencement of exercise. In certain embodiments the composition comprises about 300 mg caffeine and about 200 mg L-theanine per serving.

According to another aspect, methods and compositions for improving exercise performance in individuals tolerant to the effects of caffeine due to regular daily caffeine consumption are provided. In certain embodiments the composition is administered from about 30 minutes to 45 minutes prior to the commencement of exercise. In certain embodiments the composition comprises about 300 mg caffeine and about 200 mg L-theanine per serving.

According to another aspect, methods and compositions for improving recovery from exercise in individuals tolerant to the effects of caffeine due to regular daily caffeine consumption are provided. In certain embodiments the composition is administered from about 30 minutes to 45 minutes prior to the commencement of exercise. In certain embodiments the composition comprises about 300 mg caffeine and about 200 mg L-theanine per serving.

According to another aspect, methods for maintaining the efficacy of a caffeine-containing composition administered prior to the commencement of exercise are provided. In certain embodiments the composition is administered from about 30 minutes to 45 minutes prior to the commencement of exercise. In certain embodiments the composition comprises about 300 mg caffeine and about 200 mg L-theanine per serving.

### DETAILED DESCRIPTION OF THE EMBODIMENTS

The present invention is directed towards pre-workout supplementation with caffeine and L-theanine. According to various embodiments, methods for improving exercise performance are provided. According to other embodiments, methods for improving recovery from exercise are provided. According to other embodiments, methods for improving exercise performance in individuals tolerant to the effects of caffeine due to regular daily caffeine consumption are provided. According to other embodiments, methods for improving recovery from exercise in individuals tolerant to the effects of caffeine due to regular daily caffeine consumption are provided. According to other embodiments, methods for maintaining the efficacy of a caffeine-containing composition administered prior to the commencement of exercise are provided. The compositions employed in the various methods herein provided comprise caffeine and theanine in a ratio of 3:2 (weight/weight). In preferred embodiments, the amount of caffeine is about 300 mg per serving, and the amount of L-theanine is about 200 mg per serving. The compositions are preferably administered from about 30 minutes to 45 minutes prior to the commencement of exercise.

The present compositions and methods and their various embodiments may be of use to both habitual caffeine consumers and non-habitual. Although there is no specific widely accepted definition of a ‘habitual’ caffeine consumer, overall the literature supports amounts of over 100 mg per day as defining habitual caffeine use. It is herein understood that most habitual caffeine consumers, as above identified, will have some degree of tolerance, real or perceived, to the effects of caffeine.

Study 1

The inventors, having experience in the efficacy of various dosages of caffeine in the context of pre-workout supplements in both habitual and non-habitual caffeine users, first examined a pre-workout composition containing 250 mg caffeine anhydrous and 200 mg L-theanine. In this first study, twenty-one subjects (20 male, 1 female), all healthy and between the ages of 20 and 35 years were given the pre-workout supplement in capsule form to be taken 30 to 45 minutes before exercise on an empty stomach in place of any regularly consumed pre-workout product prior to the next exercise session. Subjects were instructed to maintain normal diet and activity. Subjects were surveyed for ratings of perceived effects on energy levels and mental focus. Subjects were also advised to supply general comments regarding the supplement in terms of negative side effects, comparison to other products, overall satisfaction and effectiveness. Only 43% were satisfied with the results and felt that the experimental composition was superior to caffeine alone in providing energy during exercise, and 71% were unsatisfied and said they would like to feel a more powerful stimulant effect. Therefore, a subsequent experiment was conducted with 50 mg more caffeine added to determine the optimal dose for enhanced energy and focus during workouts.

Study 2

In a subsequent study 18 subjects were given a pre-workout supplement containing 300 mg caffeine anhydrous and 200 mg L-theanine. Subjects were instructed and monitored as described above in Study 1. Treatment dose was well tolerated by all the participants and 66% were satisfied with the boost in energy and focus and felt this combination was superior to the previous sample (where only 43% were satisfied). Also, surprisingly, only 11% expressed the desire for a greater stimulant effect, in contrast to the 71% found in Study 1.

Most subjects in Study 2 reported an overall improvement in alertness, yet feeling slightly more relaxed than caffeine alone. In addition, L-theanine prevented jitters and the energy crash that participants typically experience with high amounts of caffeine. Thus, it appeared that the addition of 200 mg of L-theanine to 300 mg of caffeine, taken 30-45 minutes prior to exercise, improved or at least maintained the efficacy of the composition relative to caffeine alone in most subjects.

The inventors believe that a 3:2 weight ratio of caffeine-to-L-theanine to provide optimal effects in terms of pre-workout supplementation to the average individual. Of course, the amounts of caffeine and L-theanine will depend on a number of variables including body weight and the regular daily intake of caffeine. However, it is believed that 300 mg of caffeine and 200 mg of L-theanine will be suitable for the average individual that consumes caffeine daily (e.g. at least 100 mg per day). For individuals that do not consume 100 mg per day of caffeine, it is recommended to determine sensitivity to the composition at a lower amount of caffeine while maintaining the 3:2 caffeine-to-L-theanine ratio.

Thus, in one aspect there is provided a composition and method for improving upon the usefulness of caffeine as an ergogenic aid. Such methods comprise administration of a composition comprising caffeine and theanine in a ratio of approximately 3:2 caffeine to theanine. In particular embodiments, the composition comprises approximately about 60 to about 300 mg of theanine per serving; preferably from about 150 to 375 mg of caffeine per serving, and from about 100 to 250 mg theanine, maintaining a ratio of approximately 3:2 caffeine to theanine. In a preferred embodiment, the composition comprises approximately 300 mg of caffeine and approximately 200 mg of L-theanine.

In another aspect there is provided a composition and method to reduce some of the unwanted effects associated with the pre-workout consumption of caffeine and while maintaining or improving upon its usefulness as an ergogenic aid. Such methods comprise administration of a composition comprising caffeine and theanine in a ratio of approximately 3:2 caffeine to theanine. In particular embodiments, the composition comprises approximately about 60 to about 300 mg of theanine per serving; preferably from about 150 to 375 mg of caffeine per serving, and from about 100 to 250 mg theanine, maintaining a ratio of approximately 3:2 caffeine to theanine. In a preferred embodiment, the composition comprises approximately 300 mg of caffeine and approximately 200 mg of L-theanine.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a caplet or capsule or as a beverage, either ready-to-drink or as a powder beverage mix.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

Still further, the compositions and methods herein described may be suitable for use with additional ingredients, particularly ingredients commonly found in pre-workout supplements. Such ingredients include, but are not limited to: arginine, citrulline, tyrosine, creatine, carnosine, beta-alanine, taurine, vinpocetine, xanthinol nicotinate, vitamin C, niacin/vitamin B3, folic acid/vitamin B9, vitamin B6, vitamin B12, or salts thereof; yohimbine, evodiamine, carbohydrates, or sources thereof; Citrus Aurantium, Rhodiola Rosea, Rhodiola Crenulata, Panax Ginseng, Eleutherococcus senticosus, Schisandra Chinensis, or extracts thereof.

The preferred form of caffeine is caffeine anhydrous; however, the source of caffeine may be other known sources such as Coffea Arabica, Coffea canephora, Camellia sinensis, Ilex paraguariensis, Paullinia cupana, Theobroma cacao, and kola nut, provided the required amount of caffeine is provided.

L-theanine may be obtained from known methods such as extraction from tea leaves, fermentation, chemical synthesis, or by biosynthesis using plants or microorganisms.

### Example 1

A pre-workout supplement to provide energy during exercise, while preventing feelings of jitteriness and an energy crash. The supplement is provided in the form a capsule and contains 300 mg caffeine anhydrous and 200 mg L-theanine per serving. One serving is to be consumed with water 30-45 minutes prior to exercise.

### Example 2

A pre-workout supplement to provide energy during exercise, while preventing feelings of jitteriness and an energy crash, for use by an individual unaccustomed, or sensitive, to caffeine. The supplement is provided in the form a capsule and contains 150 mg caffeine anhydrous and 100 mg L-theanine per serving. One serving is to be consumed with water 30-45 minutes prior to exercise.

The above-described embodiments are intended to be examples of the present invention. Those of skill in the art will recognize that alterations and modifications may be effected thereto, without departing from the scope of the invention which is defined solely by the claims appended hereto.

What is claimed is:

1. A method for improving exercise performance comprising administering to an individual a composition prior to the exercise, the composition comprising caffeine and L-theanine in a ratio of about 3:2.

2. The method of claim 1, wherein the composition is administered from about 30 minutes to 45 minutes prior to the commencement of exercise.

3. The method of claim 1, wherein the composition comprises about 300 mg caffeine and about 200 mg L-theanine per serving.

4. A method for improving recovery from exercise comprising administering to an individual a composition prior to the exercise, the composition comprising caffeine and L-theanine in a ratio of about 3:2.

5. The method of claim 4, wherein the composition is administered from about 30 minutes to 45 minutes prior to the commencement of exercise.

6. The method of claim 4, wherein the composition comprises about 300 mg caffeine and about 200 mg L-theanine per serving.

7. A method for improving exercise performance in individuals tolerant to the effects of caffeine due to regular daily caffeine consumption, the method comprising administering to an individual a compostion comprising about 300 mg caffeine and about 200 mg L-theanine.

8. The method of claim 7, wherein the compostion is administered from about 30 minutes to 45 minutes prior to the commencement of exercise.

9. A method for improving recovery from exercise in individuals tolerant to the effects of caffeine due to regular daily caffeine consumption, the method comprising administering to an individual a composition comprising about 300 mg caffeine and about 200 mg L-theanine.

10. The method of claim 9, wherein the compostion is administered from about 30 minutes to 45 minutes prior to the commencement of exercise.

11. A method for maintaining the efficacy of a caffeine-containing composition administered prior to the commencement of exercise, the method comprising administering to an individual a composition comprising caffeine and L-theanine in a ratio of about 3:2.

12. The method of claim 11, wherein the compostion is administered from about 30 minutes to 45 minutes prior to the commencement of exercise.

13. The method of claim 11, wherein the composition comprises about 300 mg caffeine and about 200 mg L-theanine per serving.
